(19) United States
(12) Patent Application Publication
(10) Pub. No.: US 2005/0222263 A1
(43) Pub. Date: Oct. 6, 2005

(54) Compositions against stress-related diseases and methods for treating stress-related diseases

(75) Inventors: Miroslav Smriga, Kawasaki-shi (JP); Kazuyoshi Takahashi, Kawasaki-shi (JP); Yasushi Morinaga, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 11/082,664

(22) Filed: Mar. 18, 2005

Related U.S. Application Data
(63) Continuation of application No. PCT/JP03/11754, filed on Sep. 16, 2003.

(30) Foreign Application Priority Data
Sep. 18, 2002 (JP) 2002-271944

(51) Int. Cl. A61K 31/198

(52) U.S. Cl. 514/564

(57) ABSTRACT

Administering or ingesting a combination of lysine with another agent for stress-related diseases other than lysine provides an improvement in the treatment and prevention of stress-related diseases (enhanced drug efficacy) and reduced side effects. Such combinations of lysine with another agent for stress-related diseases other than lysine can be employed in the form of a medicine (including a medicine for animals), a food, a drink or a feed, and are highly useful in preventing, treating, or ameliorating various stress-related diseases and preventing the progress of such diseases. The two ingredients as described above can be administered or ingested either at the same time or at different points and in different forms.

### CROSS REFERENCES TO RELATED APPLICATIONS

This application is a continuation of International Patent Application No. PCT/JP03/11754, filed on Sep. 16, 2003, and claims priority to Japanese Patent Application No. 2002-271944; filed on Sep. 18, 2002, both of which are incorporated herein by reference in their entireties.

### BACKGROUND OF THE INVENTION

1. Field of the Invention

The present invention relates to compositions for treating stress-related diseases. These compositions when used as a drug are very useful in the treatment, prevention, prevention of development, and/or improvement of stress-related diseases in animals such as humans.

The present invention also relates to a method for the treatment or prevention (including treatment, prevention, prevention of development, and improvement) of a stress-related disease and to the use of the above-mentioned active ingredient as a drug (including a drug for treatment, prevention, prevention of development, and improvement) for the treatment or prevention of a stress-related disease.

2. Discussion of the Background

Stress affects various general organs such as circulatory organs and digestive organs to cause stress-related diseases such as depression, anxiety, gastro-duodenal ulcer, irritable bowel syndrome, bronchial asthma, hypertension, autonomic imbalance, and the like. In a case of depression, the employed basic therapeutic principles are: “taking sufficient rest,” “using an appropriate anti-depressant,” and “supportive psychotherapy like cognitive therapy.” Though tricyclic or tetracyclic anti-depressants have so far been used in the drug therapy, they are accompanied by side effects, such as dry mouth, constipation, dysuria, disorder of eye control, and the like, making their use difficult. Recently, selective serotonin reuptake inhibitors (SSRIs) and selective serotonin/norepinephrine reuptake inhibitors (SNRIs), which allow decrease of adverse reactions, have been proposed, but the side effect of gastric mucosa disorder has been observed, and it is desired to avoid such a side effect.

In another stress-related disease, irritable bowel syndrome, a certain drug therapy has been made in addition to psychotherapy and dietary therapy. In the drug therapy, however, a sufficient effect has not yet been attained, though an anti-cholinergic agent, anti-diarrheal agent or laxative as well as a minor tranquilizer has been used mainly for the relaxation of the disease condition (abdominal pain, diarrhea, constipation, etc.). Thus, a highly effective therapeutic agent is desired.

Thus, there are several problems in the treatment (therapy) of stress-related diseases with drugs, that is: (1) insufficient effect; and (2) occurrence of adverse reactions to reduce the usefulness. In order to solve or improve these problems, a variety of studies have been attempted up to now, but no satisfactory therapeutic method has been achieved.

Thus, there remains a need for an anti-stress agent in which the above-mentioned problems have been improved. There also remains a need for improved methods for treating and preventing stress-related diseases.

### SUMMARY OF THE INVENTION

Accordingly, it is one object of the present invention to provide novel compositions for treating and preventing stress-related diseases.

It is another object of the present invention to provide novel methods for treating and preventing stress-related diseases.

It is another object of the present invention to provide novel anti-stress agents (a drug effective to a stress-related disease), for example, a drug in which the effect (drug efficacy) is enhanced more than that of the conventional drugs with lesser adverse reaction.

It is another object of the present invention to provide novel combinations of lysine with a drug for stress-related diseases, other than the lysine, (i.e., a drug exhibiting an anti-stress effect) administrable at the same time or separately as a composition (drug, drug for animal use, food, drink, feed, etc.) or a drug (drug exhibiting an anti-stress effect) for treating stress-related diseases.

These and other objects, which will become apparent during the following detailed description, have been achieved by the inventors' discovery that compositions prepared by blending one of amino acids, lysine, with an anti-stress agent (drug for stress-related diseases) or a combination of lysine with such an agent exhibits an enhanced effect (drug efficacy) and reduced adverse reaction in comparison with the anti-stress agent per se containing no lysine.

In this connection, recently, some of the present inventors found that one of essential amino acids, lysine, has anti-stress effect; this finding has been filed as a patent application PCT/JP02/02571. Therefore, since lysine corresponds to one of anti-stress agents, lysine and another anti-stress agent are employed as active ingredients.

In the present invention, it is considered that there is a possibility of lysine enhancing the effect of the drug used as a therapeutic for the above-mentioned diseases, resulting in a decrease of the incidence of side effects by reduction of the dosage of the drug. Thus, the combined use of lysine increases synergistically or additively the effect of the anti-stress agents. In particular, among the drugs so far used in treatment of stress-related diseases, those of which the effect is insufficient in single use or the expected efficacy is reduced by their side effect, are expected to contribute to the treatment (therapy) of these stress-related diseases as medical or food stuffs since their usefulness is increased in combination with lysine.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows the combined effect of lysine and a minor tranquilizer alprazolam in an irritable bowel syndrome model (Example 1), *: significant in comparison with a control (p<0.05); and

FIG. 2 shows a reducing effect of lysine for exacerbation of intragastric hemorrhage by SSRI in a gastric ulcer model (Example 2), SSRI: paroxetine, *: significant in comparison with a control (p<0.05).

### DETAILED DESCRIPTION OF THE PREFERRED EMBODIMENTS

Thus, in a first embodiment, the present invention provides novel compositions for treating stress-related diseases which comprise at least lysine and another anti-stress agent other than lysine as active ingredients (a composition of the invention).

The lysine may be used in a form of one or more salts such as the hydrochloride and the glutamate. The lysine may be used in any form of the optical isomers including D-isomer, L-isomer, and DL-isomer; preferably, the L-isomer is used in the view that it is naturally occurring. The subjects to be treated with the anti-stress agent include humans (a patient or a person who needs the prevention) as well as animals which have a stress-related disease or possibly suffering from such a disease, for example, a domestic animal.

The above-mentioned composition may be used in the form of a drug (including animal drugs), a food or drink, and a feed.

As for the above-mentioned anti-stress agents, one or more of the following drugs (1) to (3) may be selected:

(1) drugs acting on the neurotransmission system participating in the central and autonomic nervous system;

(2) drugs acting on the immune system; and

(3) drugs which relieve a disease condition by a mechanism other than the neurotransmission system and immune system.

The above-mentioned anti-stress agents, one or more, may be selected from the following drugs: anti-depressants, anti-anxiety agents, agents for treating irritable bowel syndrome, agents for treating gastro-duodenal ulcer, agents for treating bronchial asthma, anti-hypertensive agents, agents for treating angina pectoris, anti-diabetics, and anti-rheumatism agents.

The above anti-depressants include selective serotonin reuptake inhibitors and selective serotonin/norepinephrine reuptake inhibitors; the above anti-anxiety agents include benzodiazepine derivatives, diphenylmethane derivatives, and drugs acting on serotonin 5-HT1A receptors; the above agents for treating irritable bowel syndrome include anti-cholinergic agents, anti-diarrheal agents, and laxatives; the above agents for treating gastro-duodenal ulcer include histamine H2 receptor antagonists, proton pump inhibitors, and prostaglandin-type agents; the above agents for treating bronchial asthma include adrenaline β2 receptor agonists and anti-histaminics; the above anti-hypertensive agents include Ca-channel antagonists, angiotensin converting enzyme inhibitors, angiotensin II receptor antagonists, and diuretics; the above agents for treating angina pectoris include organic nitric acid esters and adrenaline β receptor blockers; and the above anti-diabetics include insulin secretion promoters, insulin preparations, and sulfonylureas.

The above-mentioned drugs are exemplified by anti-stress agents (drugs with an anti-stress effect) and can be used in the treatment, prevention, improvement, and prevention of the development of such diseases.

There is no particular limitation in the ratio of the above-mentioned anti-stress agent (active ingredient) to lysine to be used. The ratio (by weight) of the anti-stress agent to lysine when calculated in the free form of lysine is preferably in the range of approximately 1:0.5 to 1:100000, more preferably 1:1 to 1:10000, and even more preferably 1:2 to 1:5000.

The above-mentioned composition of the present invention comprises at least lysine and an anti-stress agent other than lysine (one or more of active ingredients), and further it may contain other ingredients as far as they do not adversely affect the purpose of the invention. For example, an amino acid other than lysine may be used as an additive. For example, arginine, glutamic acid, aspartic acid, and so on may be added.

In another embodiment of the invention, the anti-stress agent includes a combination of lysine with an anti-stress agent other than lysine which may be administered at the same time or separately (a combination of the invention).

In the same manner as in the above invention, the lysine may be used in a form of one or more salts such as the hydrochloride, glutamate, and the like. The lysine may be used in any form of the optical isomers including D-isomer, L-isomer and DL-isomer; preferably, the L-isomer is used since it is naturally occurring. The subject to be treated with the anti-stress agent includes humans as well as animals possibly suffering from stress-related diseases, for example, domestic animals.

The combination of the invention includes the above composition of the invention as a form for concurrent administration of the above two active ingredients. The description of the above-mentioned composition of the invention applies to the combination of the present invention except for that of “composition” per se.

The combination of the invention may be used in any form including the above-mentioned drugs (including drugs for animals), a food, a drink, and a feed. In particular, the anti-stress agents may be administered as a drug or a drug for animal use (single preparation); on the other hand, lysine may be given (administration, diet, or supply) in a form of a drug, a food, a drink, or a feed (single preparation of lysine, drink containing lysine, feed containing lysine, etc.), respectively at a different point in time. In this operation, it is naturally possible that they may contain additional ingredients necessary for the drug (including a drug for animal use), food, drink, or feed, or they may further be processed if necessary.

In another embodiment, the present invention provides a method for preventing or treating a stress-related disease (including treatment, prevention, prevention of development, improvement, etc.), which comprises ingesting or administering lysine and an anti-stress agent other than lysine to the living body. The lysine may be used in the form of one or more salts.

The ingestion or administration may be made by ingesting or administering the above-mentioned composition or in a form of the above-mentioned combination of the invention.

Still in another embodiment, the invention relates to the use of (or the preparation of) the drugs for preventing or treating stress-related diseases (including treatment, prevention, prevention of development, etc.) comprising lysine and an anti-stress agent other than lysine. Once again, the lysine may be in a form of one or more salts.

The stress-related diseases are as described above in the present description. As for the drugs for preventing or treating stress-related diseases, the above-mentioned composition or combination of the invention or one further containing the above-mentioned ingredients may be exemplified as a preferred embodiment.

The combination of the present invention as mentioned above can be used in the form of a drug (including drugs for animal use), a food, a drink, or a feed. In this invention, the two active ingredients, i.e., lysine and the anti-stress agent (active ingredient), each can be used separately in a different form. Hereinafter, a representative formulation of the invention will be explained as an example of the products (pharmaceutical preparations) which contain the above two active ingredients in the same preparation, though it is just a typical example and the invention is not limited thereto.

The term stress-related diseases in the context of the invention mean those caused by any stress (see: “Karada no Kagaku” (Body Science), vol. 223, pp. 58-61 (2002)), and will be explained in detail by the followings.

The autonomic nervous system and immune system are affected by stress to cause the following stress-related diseases. The diseases include depression or anxiety in the psychic nervous system; gastro-duodenal ulcer or irritable bowel syndrome in the digestive organ system; bronchial asthma or hyperventilation syndrome in the respiratory system; hypertension or angina pectoris in the circulatory system; diabetes mellitus in the endocrine system; migraine headache or autonomic imbalance in the neuromuscular system; and chronic articular rheumatism or collagen disease in the immune and allergy system.

The drugs used in these diseases can roughly be classified into three groups. The first group includes drugs acting on the neurotrasmission system participating in the central and autonomic nervous systems which are activated by stress per se. The second group includes drugs acting on the immune system altered by stress, and the third group includes drugs for improving (alleviating) the condition per se recognized in the respective diseases which are accompanied by alteration of the above neurotransmission system and immune system.

The effect of lysine on stress-related diseases is considered to occur as an effect on the serotonin nervous system and the modification of a benzodiazepine receptor.

A drug for which the effect is expected to increase in combination with lysine may be, for example, a conventional drug or a drug which may be developed in future, includes preferably those of which the action mechanism is different from that of lysine. That is, it includes: (1) those drugs which act on the neurotransmission system participating in the central and autonomic nervous system, which has the site of action different from that of a serotonin nervous system and benzodiazepine receptor in which lysine is possibly involved; (2) those drugs which act on the immune system; and (3) those drugs which alleviate the condition based on a mechanism other than a neurotransmission system and immune system.

Concrete examples are: (1) the group of drugs which act on the neurotransmission system participating in the central and autonomic nervous system, including those SSRIs which have recently been used as an anti-depressant, e.g., paroxetine and fluvoxamine, SNRIs, e.g., milnacipran; benzodiazepine derivatives as anti-anxiety agents, e.g., diazepam, oxazepam, and flutoprazepam; diphenylmethane derivatives, e.g., hydroxyzine; and serotonin 5-HT1A receptor agonists, e.g., tandospirone; as well as other drugs controlling neurotransmission (catecholamine, serotonin, acetylcholine, neuropeptides, etc.); adrenaline β-receptor blockers, e.g., propranolol, pindolol, and timolol; adrenaline α1-receptor blockers, e.g., prazosin and bunazosin; adrenaline α,β-receptor blockers, e.g., labetalol; muscarine receptor antagonists, e.g., tiquizium and trospium; serotonin 5-HT3 receptor antagonists, e.g., alosetron; and serotonin 5-HT4 receptor agonists, e.g., tegarerod;

(2) the group of drugs which act on the immune system and which includes non-specific immunoactivators, e.g., lentinan and schizophyllan; interleukin production inhibitors, e.g., cyclosporin, suplatast, and tazanolast; and

(3) the group of drugs which alleviate the condition based on a mechanism other than the neurotransmission system and the immune system. Specific drugs which act on gastro-duodenal ulcers include, for example, histamine H2 receptor antagonists which directly inhibit the secretory mechanism of an aggressive factor, i.e. acid, e.g., famotidine, cimetidine, ranitidine, and nizatidine; and proton pump inhibitors, e.g., lansoprazole, omeprazole, and rabeprazole. Drugs for treating irritable bowel syndrome include those which improve constipation, e.g., polycarbophil calcium, methylcellulose, and ragnolose. The antihypertensive agents include Ca-channel antagonists which directly relax the vascular smooth muscle to decrease blood pressure, e.g., cilnidipine, amlodipine, and efonidipine; angiotensin converting enzyme inhibitors which directly inhibit the hypertensive factor renin-angiotensin system, e.g., enalapril, captopril, and temocapril; and angiotensin II receptor antagonists, e.g., losartan, valsartan, and candesartan cilexetil. The drugs for treating bronchial asthma include adrenaline β2 receptor agonists, e.g., procaterol, trimethoquinol, hexoprenaline, and salbutamol; the drugs for treating diabetes mellitus include those which directly control insulin secretion, e.g., tolbutamide or nateglinide, glibenclamide, and chlorpropamide.

In using the anti-stress agents as mentioned above, they may be given in any conventional way corresponding to the respective drugs; the dose of the active ingredient may be determined in the conventional way, which may be properly increased or decreased in consideration of the effect of the invention (dose of approximately 10-80%). In other words, as a result of the inclusion of lysine in the present combinations, compositions, and methods, the dosage of the other anti-stress agent may typically be reduced to 10 to 80% of the amount in which it is conventionally administered.

Therefore, it is considered that the occurrence of side effect caused by these drugs could be reduced by decreasing the dose with the effect enhanced by a combined use of lysine.

The lysine used in combination with a drug for treating stress-related diseases can be incorporated into the above pharmaceutical preparations. Of course, it may be used alone or as a pharmaceutical composition prepared by blending with any known pharmaceutically acceptable carrier, diluent, and the like. In such a case, there is no particular limitation in a way of administration, and it may be used as an oral preparation such as tablets, capsules, powders, granules, pills, and the like, or as a parenteral preparation such as injections, syrup, ointment, suppositories, and the like. The dose of lysine is variable depending on the subject to be administered, the route of administration, and conditions; it may be administered preferably at a daily dose of about 10 mg to 50 g, and more preferably about 100 mg to 20 g, at once or in several divided doses a day regardless of its formulation as lysine alone or as a combination drug.

The present invention, as mentioned above, also relates to a method for preventing or treating stress-related diseases (including treatment, prevention, prevention of development, improvement, etc.), which comprises ingesting or administering lysine and an anti-stress agent other than lysine to the living body, or alternatively the use of (or in preparation of) a drug for preventing or treating stress-related diseases (including treatment, prevention, prevention of development, improvement, etc.), which comprises lysine and an anti-stress agent other than lysine. In these cases, the lysine may be in the form of one or more salts.

The present invention can readily be carried out based on the above explanation of the composition of the invention and a combination of the invention or the working examples as mentioned below, or if necessary by referring to a conventional known technology.

Other features of the invention will become apparent in the course of the following descriptions of exemplary embodiments which are given for illustration of the invention and are not intended to be limiting thereof.

### EXAMPLES

### Example 1

### Effect of the Combined Use of Alprazolam and Lysine in an Animal Model for Irritable Bowel Syndrome

Method

An animal model of irritable bowel syndrome was prepared according to the method as described by Williams et al. (Williams, C. L., Villar, R. G., Peterson, J. M., Burks, T. F., “Stress-induced changes in intestinal transit in the rat: a model for irritable bowel syndrome,” Gastroenterol., vol. 94, pp. 611-621 (1988)). That is, a cotton tape was placed around the anterior limbs of seven-week-old Wistar male rats, the rats were kept in a bracket cage, and then the amount of feces excreted during 2 hours was measured. A test compound was orally administered 40 minutes before starting the wrap-restraint stress. The following groups were examined: 1) control group; 2) a group to which lysine hydrochloride (1 g/kg) alone was given; 3) a group to which a minor tranquilizer alprazolam (10 mg/kg; Takeda Chem. Ind.) alone was given; and 4) a combination group to which lysine hydrochloride (1 g/kg) and alprazolam (10 mg/kg) were given. Each group consisted of 10 animals.

Results

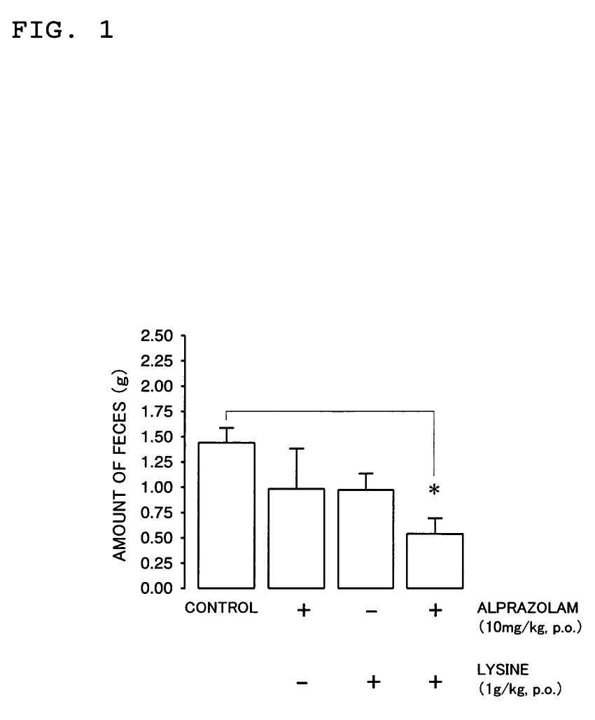

The results are presented graphically in FIG. 1. The amount of feces excreted over 2 hours during which time wrap-restraint stress was in force was about 1.4 g for the control group; this value was significantly higher than that of no stress. In comparison with this increase induced by stress, no clear effect was observed in the group to which lysine hydrochloride or alprazolam alone was given. In the group to which a combination of lysine and alprazolam was given, however, the increase of feces was significantly inhibited (see FIG. 1). That is, the results in FIG. 1 indicate that lysine acts to increase the effect of the minor tranquilizer alprazolam.

### Example 2

### Effect of the Combined Use of SSRI and Lysine in a Model for Gastric Ulcer

Method

A widely used model for gastric ulcer caused by water immersion under restraint was used. That is, rats were placed in a stress cage and immersed in water to the chest for 5 hours (at a temperature of 22-25° C.). The stomach was removed and the degree of intragastric hemorrhage was observed and the hemorrhage area was calculated using an NHI image software.

The test compound was evaluated as follows. Five-week-old Wistar male rats were divided into three groups. The first group was bred with a normal powdered feed; the second group with a powdered feed to which was added a selective serotonin reuptake inhibitor (SSRI) (paroxetine; Glaxo-SmithKline; 298 mg/kg diet) alone; and the third group with a powdered feed containing the same amount of the same SSRI and lysine hydrochloride (13.3 g/kg diet) to which was additionally added arginine (13.3 g/kg diet). Each group consisted of 10 animals. Rats could freely feed before they reached 14 weeks of age. Thereafter, no feed and water were given for 2 days, and on day 3, all of the rats were subjected to the water-immersion restraint stress for 5 hours.

Results

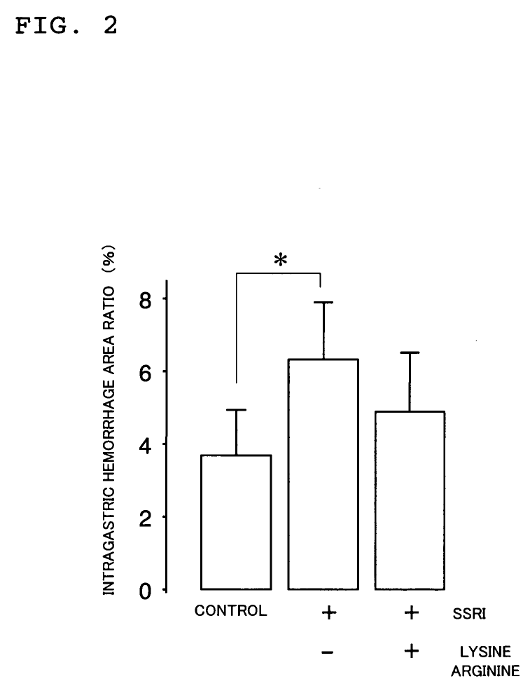

The results are shown graphically in FIG. 2. As can be seen, the SSRI significantly exacerbated the intragastric hemorrhage, but no exacerbation by the SSRI was observed in group which was fed the combination with lysine (see FIG. 2). That is, the results in FIG. 2 indicate that exacerbation of the intragastric hemorrhage by the SSRI is reduced.

From the above results, the effect of lysine combined with an anti-stress agent was confirmed.

### EFFECT OF THE INVENTION

As clearly seen from the above explanation, the present invention provides a combination of lysine and an anti-stress agent, particularly a composition comprising the two active ingredients, for example, a drug such as a combination drug (including a drug for animal use). In comparison with a single preparation of anti-stress agent, particularly a conventional anti-stress agent, the combined use of lysine exhibits an improvement of pharmacological effect and a reduction of side effects, and accordingly the products of the invention exhibit a very high efficacy. The present invention further provides a method for preventing or treating stress-related diseases (including the treatment, prevention, prevention of development, improvement, etc.) and use of the above-mentioned active ingredients in drugs for preventing or treating stress-related diseases (including the treatment, prevention, prevention of development, improvement, etc.). The present invention can widely be applied in the field of drugs (including drugs for animal use), medication, food and feed, and is very useful industrially.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A composition, comprising, as active ingredients:
(a) lysine or a salt thereof; and
(b) one or more anti-stress agents other than lysine.

2. A composition as claimed in claim 1, which is in a form selected from the group consisting of a drug, a food, a drink, and a feed.

3. A composition as claimed in claim 1, which is in a form of a drug for animal use.

4. A composition as claimed in claim 1, wherein said one or more anti-stress agents other than lysine is one or more drugs selected from the group consisting of a drug which act on a neurotransmission system participating in central and autonomic nervous systems, a drug which acts on an immune system, a drug which relieves a disease condition by a mechanism other than the neurotransmission system and immune system, and mixtures thereof.

5. A composition as claimed in claim 1, wherein said one or more anti-stress agents other than lysine is one or more drugs selected from the group consisting of an anti-depressant, an anti-anxiety agent, an agent for treating irritable bowel syndrome, an agent for treating gastro-duodenal ulcer, an agent for treating bronchial asthma, an anti-hypertensive agent, an agent for treating angina pectoris, an anti-diabetic, an anti-rheumatism agent, and mixtures thereof.

6. A composition as claimed in claim 4, wherein said anti-depressant is one or more members selected from the group consisting of a selective serotonin reuptake inhibitor, a selective serotonin/norepinephrine reuptake inhibitor, and mixtures thereof; and said anti-anxiety agent is one or more members selected from the group consisting of a benzodiazepine derivative, a diphenylmethane derivative, a drug acting on a serotonin 5-HT1A receptor and mixtures thereof.

7. A composition as claimed in claim 4, wherein said agent for treating irritable bowel syndrome is one or more members selected from the group consisting of an anti-cholinergic agent, an anti-diarrheal agent, a laxative, and mixtures thereof; and said agent for treating gastro-duodenal ulcer is one or more members selected from the group consisting of a histamine H2 receptor antagonist, a proton pump inhibitor, a prostaglandin-type agents, and mixtures thereof.

8. A composition as claimed in claim 4, wherein said agent for treating bronchial asthma is one or more members selected from the group consisting of an adrenaline β2 receptor agonist, an anti-histaminic, and mixtures thereof; said antihypertensive agent is one or more members selected from the group consisting of a Ca-channel antagonist, an angiotensin converting enzyme inhibitor, an angiotensin II receptor antagonist, a diuretic, and mixtures thereof; said agent for treating angina pectoris is one or more members selected from the group consisting of an organic nitric acid ester, an adrenaline β receptor blocker, and mixtures thereof; and said anti-diabetic is one or more members selected from the group consisting of an insulin secretion promoter, an insulin preparation, a sulfonylurea, and mixtures thereof.

9. A composition as claimed in claim 2, wherein said drug is an anti-stress agent.

10. A composition as claimed in claim 1, wherein the weight ratio of said one or more anti-stress agents other than lysine to said lysine, when calculated in a free form of lysine, is from 1:0.5 to 1:100000.

11. A composition as claimed in claim 1, which further comprises an amino acid other than lysine.

12. A combination of lysine or a salt thereof with an anti-stress agent other than lysine, each of which may be administered at the same time or separately as an anti-stress agent.

13. A method for preventing or treating a stress-related disease, which comprises ingesting or administering an effective amount of lysine or a salt thereof and an effective amount of one or more anti-stress agents other than lysine to subject in need thereof.

14. A method as claimed in claim 13, wherein said lysine or a salt thereof and said one or more anti-stress agent other than lysine are administered or ingested in a form selected from the group consisting of a drug, a food, a drink, and a feed.

15. A method as claimed in claim 13, wherein said lysine or a salt thereof and said one or more anti-stress agent other than lysine are administered or ingested in a form of a drug for animal use.

16. A method as claimed in claim 13, wherein said one or more anti-stress agents other than lysine is one or more drugs selected from the group consisting of a drug which act on a neurotransmission system participating in central and autonomic nervous systems, a drug which acts on an immune system, a drug which relieves a disease condition by a mechanism other than the neurotransmission system and immune system, and mixtures thereof.

17. A method as claimed in claim 13, wherein said one or more anti-stress agents other than lysine is one or more drugs selected from the group consisting of an anti-depressant, an anti-anxiety agent, an agent for treating irritable bowel syndrome, an agent for treating gastro-duodenal ulcer, an agent for treating bronchial asthma, an anti-hypertensive agent, an agent for treating angina pectoris, an anti-diabetic, an anti-rheumatism agent, and mixtures thereof.

18. A method as claimed in claim 17, wherein said anti-depressant is one or more members selected from the group consisting of a selective serotonin reuptake inhibitor, a selective serotonin/norepinephrine reuptake inhibitor, and mixtures thereof; and said anti-anxiety agent is one or more members selected from the group consisting of a benzodiazepine derivative, a diphenylmethane derivative, a drug acting on a serotonin 5-HT1A receptor and mixtures thereof.

19. A method as claimed in claim 17, wherein said agent for treating irritable bowel syndrome is one or more members selected from the group consisting of an anti-cholinergic agent, an anti-diarrheal agent, a laxative, and mixtures thereof; and said agent for treating gastro-duodenal ulcer is one or more members selected from the group consisting of a histamine H2 receptor antagonist, a proton pump inhibitor, a prostaglandin-type agents, and mixtures thereof.

20. A method as claimed in claim 17, wherein said agent for treating bronchial asthma is one or more members selected from the group consisting of an adrenaline β2 receptor agonist, an anti-histaminic, and mixtures thereof; said antihypertensive agent is one or more members selected from the group consisting of a Ca-channel antagonist, an angiotensin converting enzyme inhibitor, an angiotensin II receptor antagonist, a diuretic, and mixtures thereof; said agent for treating angina pectoris is one or more members selected from the group consisting of an organic nitric acid ester, an adrenaline β receptor blocker, and mixtures thereof; and said anti-diabetic is one or more members selected from the group consisting of an insulin secretion promoter, an insulin preparation, a sulfonylurea, and mixtures thereof.

21. A method as claimed in claim 14, wherein said drug is an anti-stress agent.

22. A method as claimed in claim 13, wherein said one or more anti-stress agents other than lysine and said lysine or a salt thereof are administered in a weight ratio of said one or more anti-stress agents other than lysine to said lysine or salt thereof, when calculated in a free form of lysine, of from 1:0.5 to 1:100000.

23. A method as claimed in claim 13, which further comprises administering or ingesting an amino acid other than lysine.

24. A method as claimed in claim 13, which comprises administering a combination of lysine or a salt thereof with an anti-stress agent other than lysine, each of which may be administered at the same time or separately as an anti-stress agent.
